(19) United States
(12) Patent Application Publication
(10) Pub. No.: US 2026/0232191 A1
(43) Pub. Date: Aug. 13, 2026

(54) CHANGED EVENTS VIEW

(71) Applicant: Cardiac Pacemakers, Inc., St Paul, MN (US)

(72) Inventors: David R. Will, St Paul, MN (US); Jan Hagenbrock, Rochester, MN (US); Jeffrey R. Spors, Eyota, MN (US); David R. Engebretsen, Cannon Falls, MN (US)

(21) Appl. No.: 19/532,345

(22) Filed: Feb. 6, 2026

Related U.S. Application Data
(60) Provisional application No. 63/758,140, filed on Feb. 13, 2025.

(51) Int. Cl. A61B 5/00; A61B 5/02

(52) U.S. Cl. CPC A61B 5/0022; A61B 5/02; A61B 5/7264; A61B 5/7282; A61B 5/742

(57) ABSTRACT

Systems, methods, and devices involve approaches for tracking and displaying changes made to cardiac data. Certain approaches include altering metadata associated with time series cardiac data, generating an updated table to reflect the alterations, and displaying a change indicator in a first window on a user interface based on the changes to the metadata.

### CROSS REFERENCE TO RELATED APPLICATION

This application claims priority to Provisional Application No. 63/758,140, filed February 13, 2025, which is incorporated by reference in its entirety.

### TECHNICAL FIELD

The present disclosure relates to devices, methods, and systems for monitoring, identifying, classifying, and displaying cardiac data.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes displaying time series cardiac data on a user interface and displaying prior rhythm classification indicators adjacent to the time series cardiac data. Respective locations of the prior rhythm classification indicators on the user interface are based, at least in part, on a prior table of metadata. The method further includes receiving user input via the user interface altering the metadata associated with the time series cardiac data; altering the prior table of metadata to generate an updated table; comparing the updated table to the prior table to determine changes to the metadata; displaying a change indicator in a first window on the user interface based on the changes to the metadata; and displaying an updated rhythm classification indicator adjacent to the time series cardiac data.

In Example 2, the method of Example 1, wherein the prior table and the updated table are stored to cache memory of a browser.

In Example 3, the method of Example 1 or 2, wherein the prior table and the updated table comprises a list of cardiac events associated with the time series cardiac data and start and end times for each of the cardiac events.

In Example 4, the method of any of Examples 1–3, wherein each of the cardiac events is associated with a unique identifier.

In Example 5, the method of any of Examples 1–4, wherein the change indicator is a numerical indicator representing a number of changes made to original rhythm classifications.

In Example 6, the method of any of Examples 1–4, wherein the change indicator is a numerical indicator representing a number of additions or deletions made to prior rhythm classifications.

In Example 7, the method of any of Examples 1–6, wherein the displaying the prior rhythm classification indicators occurs in a second window of the user interface, wherein the displaying the updated rhythm classification indicator occurs in a third window adjacent to the second window.

In Example 8, the method of any of Examples 1–7, further including selecting a button on the user interface to undo the altering.

In Example 9, the method of any of Examples 1–8, wherein the altering comprises adding a new rhythm event, wherein the updated rhythm classification indicator is a new rhythm classification indicator.

In Example 10, the method of any of Examples 1–8, wherein the altering comprises changing a rhythm start time or a rhythm end time, wherein the updated rhythm classification indicator is longer or shorter than one of the prior rhythm classification indicators.

In Example 11, the method of any of Examples 1–8, wherein the altering comprises changing a beat classification, wherein the updated rhythm classification indicator is a different type of cardiac event from one of the prior rhythm classifications.

In Example 12, the method of any of Examples 1–8, wherein the altering comprises changing one of the prior rhythm classifications to the updated rhythm classification.

In Example 13, a computer program product comprising instructions to cause one or more processors to carry out the steps of the method of Examples 1–12.

In Example 14, a computer-readable medium having stored thereon the computer program product of Example 13.

In Example 15, a computer comprising the computer-readable medium of Example 14.

In Example 16, a system include a remote computing system with a user interface, one or more processors, and a non-transitory computer-readable medium having a set of computer-executable instructions configured to be executed by the one or more processors to cause the remote computing system to perform one or more operations. The one or more operations including display time series cardiac data on the user interface and display prior rhythm classification indicators adjacent to the time series cardiac data. Respective locations of the prior rhythm classification indicators on the user interface are based, at least in part, on a prior table of metadata. The one or more operations further include receive user input via the user interface altering the metadata associated with the time series cardiac data, alter the prior table of metadata to generate an updated table, compare the updated table to the prior table to determine changes to the metadata, display a change indicator in a first window on the user interface based on the changes to the metadata, and display an updated rhythm classification indicator adjacent to the time series cardiac data.

In Example 17, the system of Example 16, wherein the remote computing system further includes an internet browser, wherein the prior table and the updated table are stored to cache memory associated with the internet browser.

In Example 18, the system of Example 16, wherein the prior table and the updated table comprises a list of cardiac events associated with the time series cardiac data and start and end times for each of the cardiac events.

In Example 19, the system of Example 16, wherein the change indicator is a numerical indicator representing a number of changes made to prior rhythm classifications.

In Example 20, the system of Example 16, wherein the change indicator is a numerical indicator representing a number of additions or deletions made to prior rhythm classifications.

In Example 21, the system of Example 16, wherein the prior rhythm classification indicators are displayed in a second window of the user interface, wherein the updated rhythm classification indicator are displayed in a third window adjacent to the second window.

In Example 22, the system of Example 16, wherein the prior rhythm classification indicators and the updated rhythm classification indicator are displayed in a second window of the user interface.

In Example 23, the system of Example 16, wherein the altering comprises adding a new rhythm event, wherein the updated rhythm classification indicator is a new rhythm classification indicator.

In Example 24, the system of Example 16, wherein the altering comprises changing a rhythm start time or a rhythm end time, wherein the updated rhythm classification indicator is longer or shorter than one of the prior rhythm classification indicators.

In Example 25, the system of Example 16, wherein the altering comprises changing a beat classification, wherein the updated rhythm classification indicator is a different type of cardiac event from one of the prior rhythm classifications.

In Example 26, the system of Example 16, wherein the altering comprises changing one of the prior rhythm classifications to the updated rhythm classification.

In Example 27, a method includes displaying time series cardiac data on a user interface and displaying prior rhythm classification indicators adjacent to the time series cardiac data. Respective locations of the prior rhythm classification indicators on the user interface are based, at least in part, on a prior table of metadata. The method further includes receiving user input via the user interface altering the metadata associated with the time series cardiac data; altering the prior table of metadata to generate an updated table; comparing the updated table to the prior table to determine changes to the metadata; displaying a change indicator in a first window on the user interface based on the changes to the metadata; and displaying an updated rhythm classification indicator adjacent to the time series cardiac data.

In Example 28, the method of Example 27, wherein the prior table and the updated table are stored to cache memory of a browser.

In Example 29, the method of Example 27, wherein the prior table and the updated table comprises a list of cardiac events associated with the time series cardiac data and start and end times for each of the cardiac events.

In Example 30, the method of Example 27, wherein the change indicator is a numerical indicator representing a number of changes made to prior rhythm classifications.

In Example 31, the method of Example 27, wherein the change indicator is a numerical indicator representing a number of additions or deletions made to prior rhythm classifications.

In Example 32, the method of Example 27, wherein the altering comprises adding a new rhythm event, wherein the updated rhythm classification indicator is a new rhythm classification indicator.

In Example 33, the method of Example 27, wherein the altering comprises changing a rhythm start time or a rhythm end time, wherein the updated rhythm classification indicator is longer or shorter than one of the prior rhythm classification indicators.

In Example 34, the method of Example 27, wherein the altering comprises changing a beat classification, wherein the updated rhythm classification indicator is a different type of cardiac event from one of the prior rhythm classifications.

In Example 35, the method of Example 27, wherein the altering comprises changing one of the prior rhythm classifications to the updated rhythm classification.

While multiple instances are disclosed, still other instances of the present invention will become apparent to those skilled in the art from the following detailed description, which shows and describes illustrative instances of the invention. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, a remote computer, and a user interface, in accordance with certain instances of the present disclosure.

FIG. 3 shows an example of beats that have been grouped together, in accordance with certain instances of the present disclosure.

FIG. 4 shows a portion of a user interface, in accordance with certain instances of the present disclosure.

FIG. 5 shows a block diagram depicting an illustrative method, in accordance with certain instances of the disclosure.

FIGS. 6–9 show various portions of the user interface of FIG. 4, in accordance with certain instances of the disclosure.

FIG. 10 is a block diagram depicting an illustrative computing device, in accordance with instances of the disclosure.

While the invention is amenable to various modifications and alternative forms, specific instances have been shown by way of example in the drawings and are described in detail below. The intention, however, is not to limit the invention to the particular instances described. On the contrary, the invention is intended to cover all modifications, equivalents, and alternatives falling within the scope of the invention as defined by the appended claims.

### DETAILED DESCRIPTION

Cardiac data such as electrocardiogram (ECG) data of a patient can be used to analyze and diagnose a patient’s cardiac activity and recommend treatments. To collect ECG data, one or more monitoring devices (e.g., sensors) can be coupled to the patient such that the monitoring devices sense and record the ECG data. The ECG data can be processed using one or more machine learning models, which output data such as beat classifications, rhythm (or event) classifications, heart rates, etc.

The ECG data and outputs of the machine learning model(s) can be further processed and analyzed by a human using a user interface. For example, the user interface can be used to alter the original beat classifications and the original rhythm classifications. Multiple users (e.g., via peer reviewing) may be involved in the process of analyzing ECG data and outputs of the machine learning model(s). And users such as more experienced users may desire to revert back to prior classifications to undo alterations made by less experienced users as part of a peer-review process. Certain instances of the present disclosure involve approaches for tracking and displaying changes made to cardiac data as well as approaches for undoing prior changes.

### CARDIAC MONITORING SYSTEM

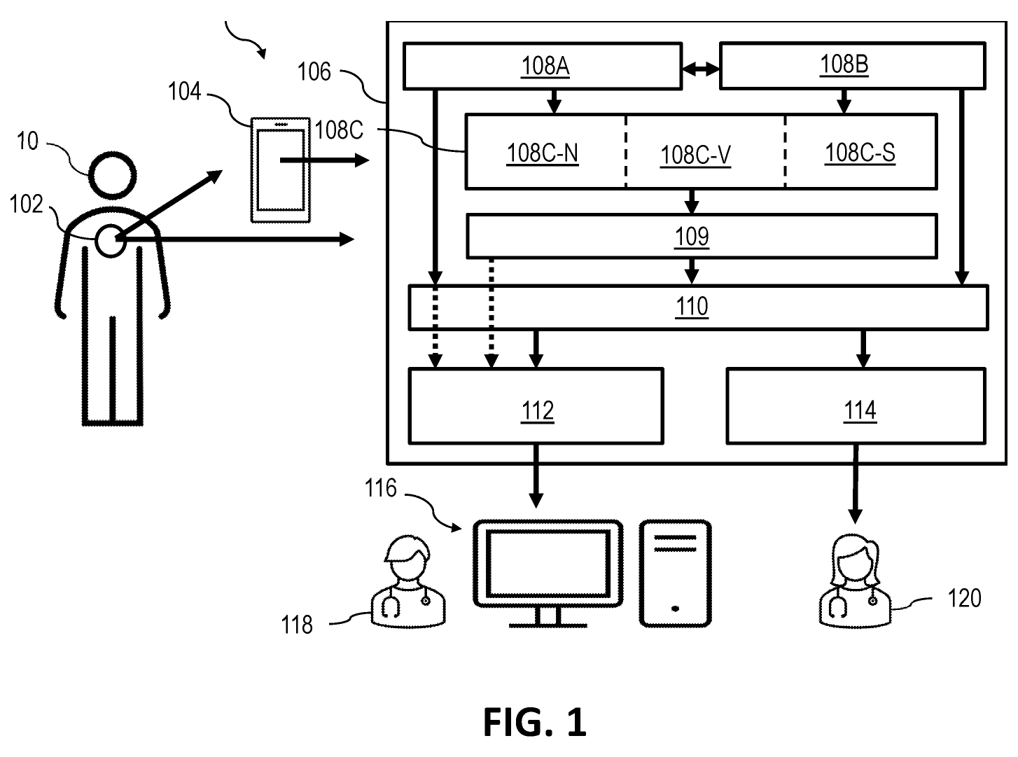

FIG. 1 illustrates a patient 10 and an example system 100. The system 100 includes a monitor 102 attached to the patient 10 or implanted in the patient 10 (e.g., pacemaker, ICD, CRT, or ICM) to detect cardiac activity of the patient 10. The monitor 102 may produce electric signals that represent the cardiac activity in the patient 10. For example, the monitor 102 may detect the patient’s heart beating (e.g., using infrared sensors, electrodes, heart sounds) and convert the detected heartbeat into electric signals representing ECG data. In certain instances, the monitor 102 stores the ECG data of a patient study (e.g., one or more days of ECG data), after which the ECG data is transmitted to another device or system such as a server. Additionally or alternatively, the monitor 102 transmits the ECG data to a mobile device 104 (e.g., a mobile phone). In such instances, the mobile device 104 can include a program (e.g., mobile phone application) that receives, processes, and analyzes the ECG data. For example, the program may analyze the ECG data and detect or flag cardiac events (e.g., periods of irregular cardiac activity) contained within the ECG data.

The mobile device 104 can periodically transmit chunks of the ECG data to another device or system such as a server, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks (e.g., an entire study’s worth of ECG data).

### CARDIAC EVENT SERVER

The ECG data (and associated metadata, if any) is transmitted to and stored by a cardiac event server 106 (hereinafter “the server 106” for brevity). The server 106 can include multiple models, platforms, layers, or modules that work together to process and analyze the ECG data such that cardiac events can be detected, filtered, prioritized, and ultimately reported to a patient’s physician for analysis and treatment. In the example of FIG. 1, the server 106 includes one or more machine learning models 108A, 108B, and 108C, a clustering algorithm module 109, a cardiac event router 110, a report platform 112, and a notification platform 114. Although only one server 106 is shown in FIG. 1, the server 106 can include multiple separate physical servers, and the various models/platforms/modules/layers can be distributed among the multiple servers. Each of the models/platforms/modules/layers can represent separate programs, applications, and/or blocks of code where the output of one of the models/platforms/ modules/layers is an input to another of the models/platforms/modules/layers. Each of the models/platforms/modules/layers can use application programming interfaces to communicate between or among the other models/platforms/modules/layers as well as systems and devices external to the server 106.

In certain instances, once the ECG data is processed by the machine learning models 108A–C and the clustering algorithm module 109, the ECG data (and associated metadata) is made available for the report platform 112. As will be described in more detail below, the report platform 112 can be accessed by a remote computer 116 (e.g., client device such as a laptop, mobile phone, desktop computer, and the like) by a user at a clinic or lab 118. In other instances, the cardiac event router 110 is used to determine what platform further processes the ECG data based on the classification associated with the cardiac event. For example, if the identified cardiac event is critical or severe, the cardiac event router 110 can flag or send the ECG data, etc., to the notification platform 114. The notification platform 114 can be programmed to send notifications (along with relevant ECG data and associated metadata) immediately to the patient’s physician/care group remote computer 116 and/or to the patient 10 (e.g., to their computer system, e-mail, mobile phone application).

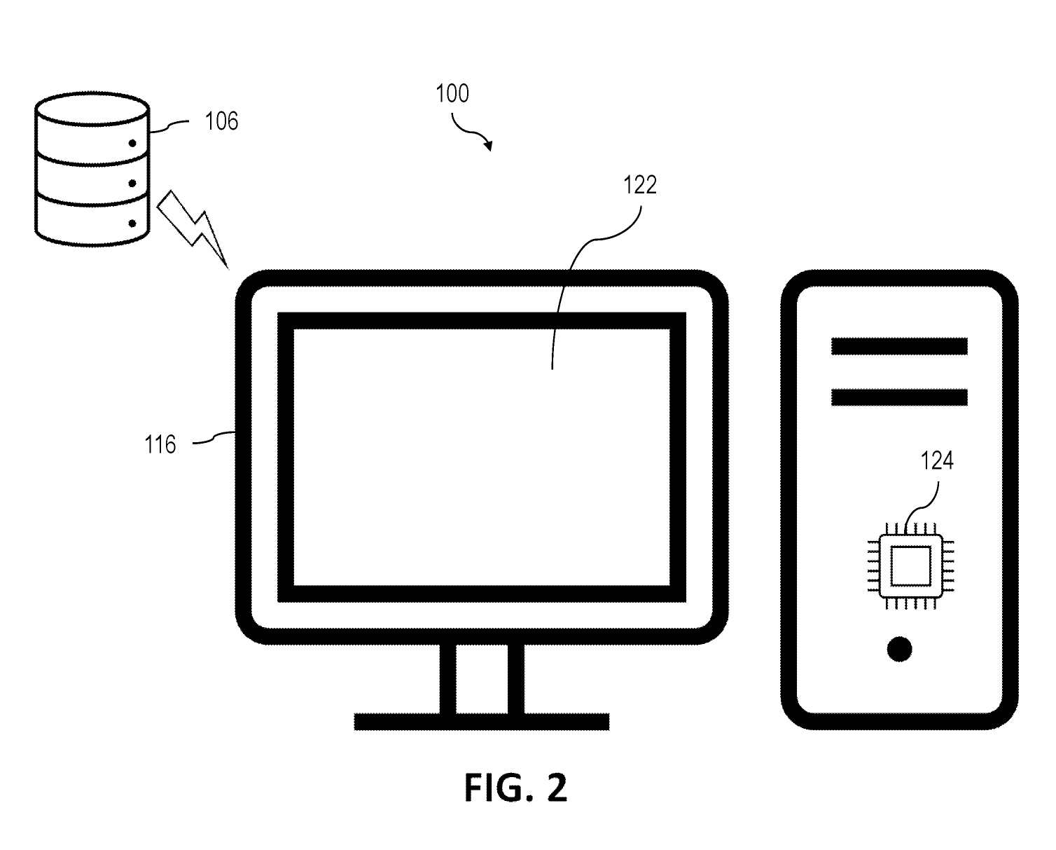

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112.

### MACHINE LEARNING MODELS

Referring back to FIG. 1, the server 106 applies the one or more machine learning models 108A–C to the ECG data to analyze and classify the beats and cardiac activity of the patient 10.

The first and second machine learning models 108A and 108B are programmed to—among other things—compare the ECG data to labeled ECG data to determine which labeled ECG data the ECG data most closely resembles. The labeled ECG data may identify a particular cardiac event—including but not limited to ventricular tachycardia, bradycardia, atrial fibrillation, pause, normal sinus rhythm, or artifact/noise—as well as particular beat classifications—including but not limited to ventricular, normal, or supraventricular beats. In addition to identifying beat classifications and event classifications (and generating associated metadata), the first and second machine learning models 108A and 108B can determine and generate metadata regarding heart rates, duration, and beat counts of the patient 10 based on the ECG data. As specific examples, the first and/or the second machine learning models 108A and 108B can identify the beginning, center, and end of individual beats (e.g., individual T-waves) such that individual beats can be extracted from the ECG data. Each individual beat can be assigned a value (e.g., a unique identifier) such that individual beats can be identified and associated with metadata throughout processing and analyzing the ECG data.

The ECG data (e.g., ECG data associated with individual beats) as well as certain outputs of the first and second machine learning models 108A and 108B can be inputted to the third machine learning model 108C. Although two machine learning models are shown and described, a single machine learning model could be used to generate the metadata described herein, or additional machine learning models could be used.

The first and second machine learning models 108A and 108B can include the neural networks described in U.S. Pat. App. No. 16/695,534, which is hereby incorporated by reference in its entirety. The first neural network can be a deep convolutional neural network and the second neural network is a deep fully-connected neural network—although other types and combinations of machine learning models can be implemented. The first machine learning model 108A receives one or more sets of beats (e.g., beat trains with 3–10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The second machine learning model 108B receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

The third machine learning model 108C (e.g., one or more trained encoder machine learning models) is programmed to generate latent space representations of the ECG data such that the ECG data is represented by fewer datapoints than the original ECG data. The latent space representations can be used as an approximation of the original raw ECG data for each beat. Although the inputs to the third machine learning model 108C are described as (1) the ECG data such as sets of individual T-waves and (2) certain outputs of the first and second machine learning models 108A and 108B, the third machine learning model 108C could be programmed to generate the latent space representations without requiring input from the first and/or second machine learning models 108A, 108B.

In certain instances, instead of a single third machine learning model 108C, the server 106 includes a separate machine learning model for each type of beat classification (e.g., normal beats, ventricular beats, and supraventricular beats). For example, as shown in FIG. 1, the server 106 may include three third machine learning models (108C-N, 108C-V, and 108C-S) instead of a single third machine learning model. In certain instances, beats that were not initially classified (e.g., unclassified beats) can be processed either by a different third machine learning model or can skip the step of generating latent space representations and being clustered with similar shaped beats.

In the example of FIG. 1, one machine learning model 108C-N is used for beats classified as normal beats, another machine learning model 108C-V is used for beats classified as ventricular beats, and another machine learning model 108C-S is used for beats classified as supraventricular beats. As such, only ECG data (e.g., T-waves) of beats initially classified as normal beats by the first and/or second machine learning models 108A, 108B—as well as metadata generated by such machine learning models—are inputted to the machine learning model 108C-N, and so on. It has been found that using machine learning models trained to focus on analyzing only certain types of beats can improve performance of the third machine learning models compared to using a single third machine learning model. Further, processing the ECG data in parallel using three machine learning models can decrease the time needed to generate the latent space representations. In certain instances, a single study may contain hundreds of thousands to millions of individual beats.

Each third machine learning model (108C-N, 108C-V, 108C-S) receives ECG data associated with individual beats (e.g., an individual clip of ECG data for each beat) and generates latent space representations of such ECG data. For example, each individual beat is processed by one of the third machine learning models—depending on each individual beat’s classification—such that the ECG data is distilled down to (or represented by) a small number of individual data points. Raw ECG data of an individual beat can include 500 or so datapoints, and each third machine learning model can distill the ECG data for a given beat into 4–16 datapoints. Put another way, each third machine learning model can generate latent space representations comprising 4–16 datapoints for a given beat. This range has been found to balance accuracy of beat representation and effectiveness of clustering (described further below). In certain instances, the latent space representations comprise 7, 8, or 9 (e.g., 7–9) datapoints for a given beat. The latent space representations comprise 1–2% of datapoints compared to the raw ECG data for each beat. Each latent space can be represented by a vector (e.g., a latent vector).

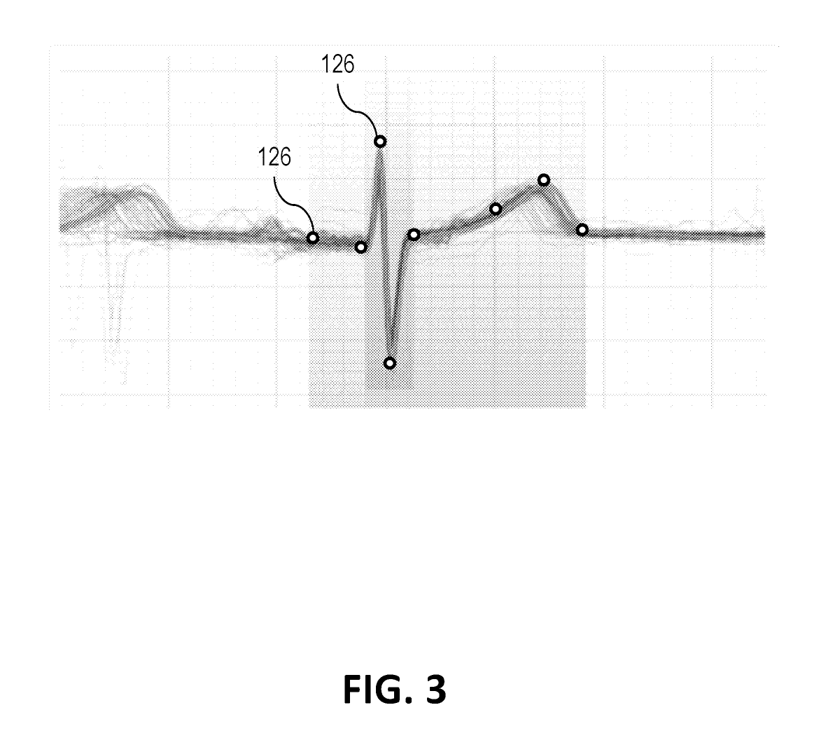

The resulting datapoints are representations of an amplitude of the ECG signal at different relative points in time. These limited datapoints are datapoints that the trained machine learning models generate such that different beat shapes can be identified and similar shaped beats can be grouped together. Put another way, these datapoints may be those that are the most likely to be helpful in distinguishing among beat shapes. The third machine learning models can leave out representations of datapoints that are less likely to help distinguish among individual beats. FIG. 3 shows an example set of beats that have been grouped or clustered together and also shows non-limiting examples of points 126 within a beat’s ECG signal that may be useful for distinguishing among beat shapes. For example, the points 126 can be located at the beginning and end of each beat, apexes (e.g., QRS peaks), nadirs, etc.

In the example of FIG. 1, the third machine learning models (108C-N, 108C-V, 108C-S) generate respective separate latent space representations for sets of beats initially classified as normal beats, ventricular beats, and supraventricular beats. In certain instances, beats that could not be initially classified (or ECG data containing artifacts due to noise) are not processed by any of the third machine learning models. Such beats can be labeled as unclassified beats.

The output(s) of the third machine learning model(s) 108C are processed by a clustering algorithm module 109. The clustering algorithm module 109 receives the latent space representations of individual beats and is programmed to associate similar shaped beats into different groups. FIG. 3 shows an example set of beats that have been grouped or clustered together. As shown in FIG. 3, ECG waveforms of individual beats (e.g., T-waves) are superimposed on each other. Each cluster or group can include hundreds or thousands of beats that have been grouped together by the clustering algorithm module 109. As can be seen, the beats all have a similar profile relative to each other. Each beat is aligned with the other beats to have respective QRS peaks centered on the graph.

In certain instances, the clustering algorithm module 109 is programmed to apply a clustering algorithm such as the k-means clustering algorithm or a derivation or variation thereof to the latent space representations. In certain instances, the same clustering algorithm module 109 and the same algorithm is used to process the latent space representations of each of the third machine learning models (108C-N, 108C-V, 108C-S). In certain instances, the output of the clustering algorithm module 109 includes assigning a value (e.g., an identifier such as a number) to each beat that is indicative of the group selected by the clustering algorithm module 109. For example, if the clustering algorithm module 109 clusters the beats into eight different groups, then all beats selected to be in the first group may be assigned a value of “1” and all beats selected to be in the second group may be assigned a value of “2” and so on. Other types of values can be used. These group values can be added to the metadata associated with each beat.

Accessing and displaying days of ECG data can be an inefficient use of computing resources, network bandwidth resources, and human resources. To help address these resource challenges, the remote computer 116 and the server 106 can communicate with each other (e.g., via commands/requests and responses) to prioritize when and what ECG data and metadata are accessible to users at the remote computer 116. The remote computer 116 can initiate commands/requests that are transmitted to the server 106, and the server 106 transmits ECG data and metadata in response to the commands/requests. For example, in some instances, the remote computer 116 receives executable code (e.g., JavaScript code) as part of an initial batch of files from the server 106, and the executable code includes code for requesting and prioritizing retrieval of data from the server 106.

The data can be downloaded in response to the remote computer 116 sending commands or requests to the server 106 for particular sets of data. For example, before raw ECG data is downloaded to the remote computer 116, the remote computer 116 can send a command or request to the server 106 for certain metadata. This metadata can include non-ECG patient data (e.g., name, physician) and pointers to ECG data and associated metadata. In certain instances, the pointers are used by the remote computer 116 to request specific strips of ECG data (e.g., specific time periods of ECG data) stored at the server 106 be downloaded to the remote computer 116. As such, to download particular strips of ECG data, the remote computer 116 can utilize the pointers to request the strips from the server 106.

The initial batch of metadata can be downloaded in a first data payload. Additionally or alternatively, the metadata initially downloaded can include beat data (e.g., classifications, locations in time, associated strip of ECG data) and cardiac event data (e.g., classifications, locations in time, associated strip of ECG data). This batch of metadata can be downloaded in a second data payload. Also, in certain instances, once a patient study session is selected, the remote computer 116 receives the executable code as described above. In other instances, the executable code is downloaded to the remote computer 116 when a user initially accesses the report platform 112 and before a patient study session is selected.

As strips of ECG data (or portions thereof) have been downloaded to the remote computer 116, the ECG and associated metadata can be displayed on the user interface 122. For example, plots of ECG data can be displayed in one or more windows of the user interface 122.

### USER INTERFACE

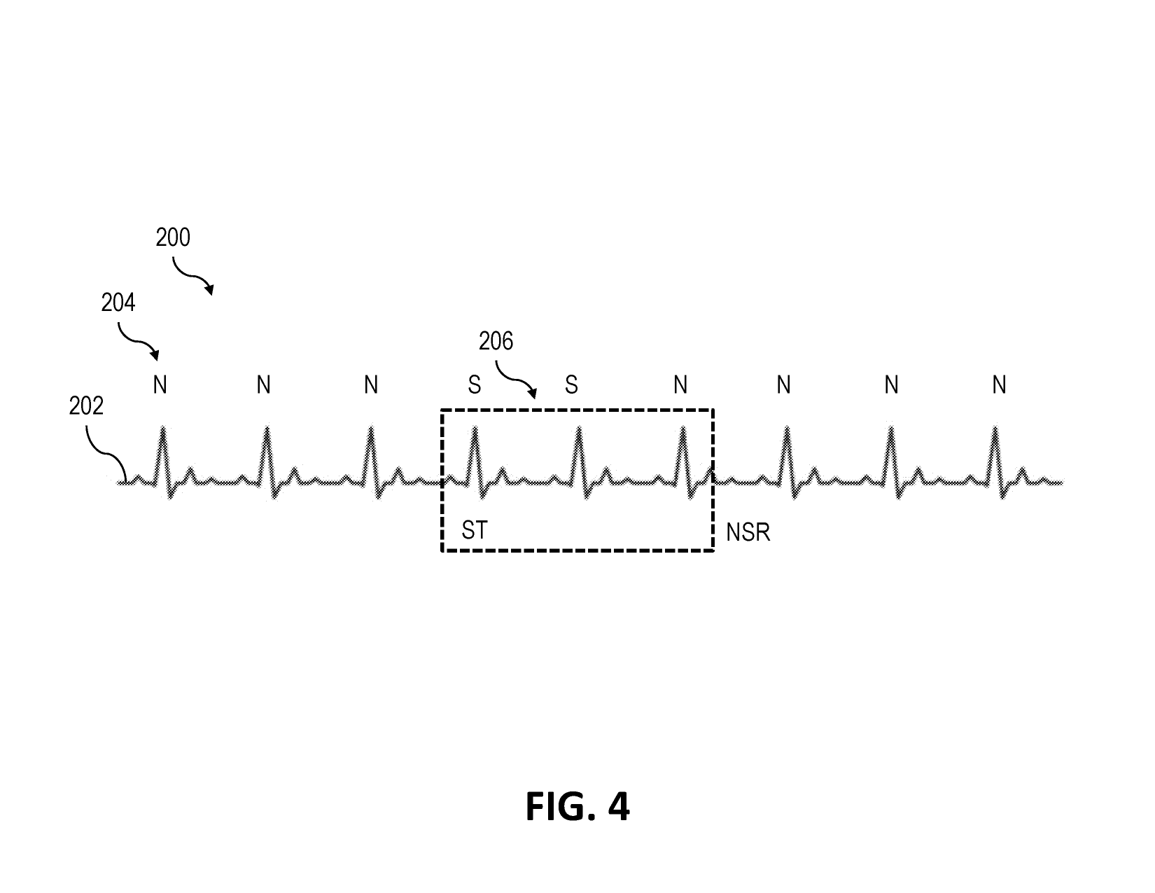

FIG. 4 shows a portion of a user interface (UI) 200. In FIG. 4, the UI 200 is displaying a plot 202 of time series cardiac data (e.g., ECG data). The UI 200 can also display metadata associated with the time series cardiac data.

As one example of displayed metadata, the UI 200 can display heartbeat classification indicators 204 for each beat that is displayed within the time series cardiac data. The original set of heartbeat classification indicators 204 can represent the beat classifications initially generated by machine learning models. The heartbeat classification indicators 204 can include a single letter such as “S” for representing supraventricular beats, “V” for representing ventricular beats, and “N” for representing normal beats. In certain instances, the heartbeat classification indicators 204 are positioned adjacent to the time series cardiac data such as immediately above a peak of each beat (e.g., immediately above the peak of the “R” wave of the QRS interval). As such, the location of the heartbeat classification indicators 204 can be based, at least in part, on the underlying metadata (e.g., the beat classification and the time at which the R wave peaked).

As another example of displayed metadata, the UI 200 can display rhythm classification indicators 206 adjacent to the time series cardiac data. The original set of rhythm classification indicators 206 can represent the rhythm classifications initially generated by machine learning models. The rhythm classifications can be classifications associated with multiple beats — as opposed to beat classifications, which can be associated with an individual beat. The rhythm classification indicators 206 can include one or more letters representing a specific type of rhythm such as “ST” for supraventricular tachycardia, “NSR” for normal sinus rhythm, “P” for pause, etc. In addition, the rhythm classification indicators 206 can include a ribbon or box, which is represented by dotted lines in FIG. 4. In certain instances, the ribbon or box is only used to indicate abnormal rhythms (e.g., rhythms other than NSR). In certain instances, the rhythm classification indicators 206 are aligned with a starting time (e.g., onset) and ending time of a given cardiac event. As such, the location of the rhythm classification indicators 206 can be based, at least in part, on the underlying metadata (e.g., the starting time and ending time of a given cardiac event).

A user can modify certain metadata using the UI 200. For example, a user can use the UI 200 to change beat classifications by selecting one or more heartbeat classification indicators 204 and changing the original beat classification (as determined by one or more machine learning models) to an updated beat classification. A user can also use the UI 200 to change the rhythm classification (or aspects thereof). For example, a user can change the original rhythm classification (as determined by one or more machine learning models) to an updated rhythm classification, can change the start time and/or end time of a cardiac event (e.g., by selecting and dragging an edge of the ribbon or box to increase or decrease the overall length), can add a new cardiac event (including a start time, end time, and rhythm classification), and can delete a cardiac event.

The metadata associated with the underlying time series cardiac data can be stored to a table. In some instances, the metadata initially generated by the one or more machine learning models is stored in an original table of metadata. The original table can store information about each beat (e.g., beat classification, time data) and each rhythm (e.g., rhythm classification, start time, end time, heart rate). In the table, each beat and each rhythm can be associated with a unique value (e.g., an alphanumerical value). For example, the table can include a list of each beat and each rhythm as well as additional information associated with each beat and each rhythm. The metadata stored to the original table (and subsequent versions of the table) can be used by the UI 200 for locations and content of heartbeat classification indicators 204 and the rhythm classification indicators 206.

Once a user modifies metadata using the UI 200, an updated table can be generated to store the updated metadata. The original table can be stored to memory (e.g., cache memory) used by an internet browser for quick access. Further, the original table can be stored at the server for more permanent storage.

Changing the classification of a cardiac event (e.g., a rhythm) can lead to automatically reclassifying beats that occurred during the event—instead of a user manually analyzing and reclassifying each of the beats. Similarly, changing the classification of one or more beats can lead to automatically reclassifying rhythms containing the changed beats—instead of a user manually analyzing and reclassifying each rhythm. Certain types of beats are typically associated with certain types of rhythms. As one example, supraventricular tachycardia events are typically associated with beats classified as supraventricular beats. As such, when a cardiac event is subsequently classified as a supraventricular tachycardia event, each of the beats occurring during that event can be reclassified to be supraventricular beats. As another example, atrial fibrillation events are typically associated with beats classified as normal beats. As such, when a cardiac event is subsequently classified as an atrial fibrillation event, each of the beats occurring during that event can be reclassified to be normal beats. This automatic reclassification of beats and/or rhythms saves time analyzing ECG data and generating summary reports while also increasing accuracy of the overall ECG studies.

Referring back to FIG. 2, to save processing and network resources and to allow these changes to metadata to occur in real-time or near-real-time, the calculations and automatic changes to the rhythm classifications and the automatic updates to the beat classifications can be carried out locally on the remote computer 116—as opposed to sending data back and forth between the server 106 and the remote computer 116. For example, the reclassifications can be carried out using cache memory 124 (shown in FIG. 1) and processing capabilities (e.g., one or more microprocessors) of the remote computer 116. To enable local processing and updating, the server 106 can send the remote computer 116 code to execute locally. This code uses (or operates on) the outputs of the one or more machine learning models such as the beat classifications and rhythm classifications (as opposed to the underlying or raw ECG data), which reduces the computational resources needed to process the changes made by user locally at the remote computer 116. In certain embodiments, this code is executed by an internet browser operating on the remote computer 116.

### CHANGED EVENTS

As noted herein, multiple users may be involved in the process of analyzing the cardiac time series data and metadata as well as making changes to the metadata. Various approaches described herein can be used to track, display, and undo changes made to the underlying cardiac data.

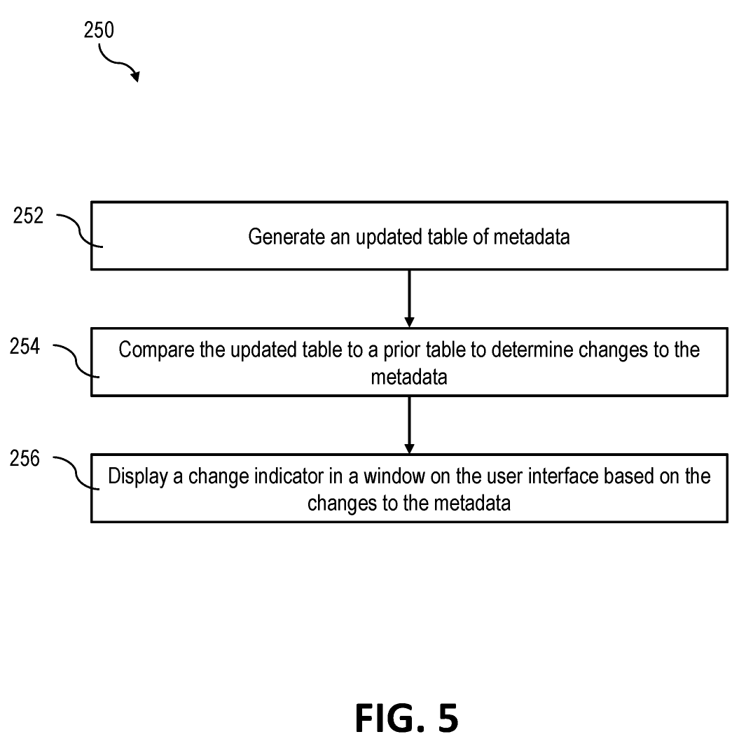

FIG. 5 outlines a method 250 for use with the UI 200. The method 250 includes generating an updated table of metadata (block 252 in FIG. 5), e.g., in response to receiving user input via the UI 200 that alters metadata associated with time series cardiac data. In certain instances, altering the metadata results in generating a separate, updated data with the as-modified metadata.

The method 250 further includes comparing the updated table to a prior version of a table of metadata (e.g., the original table of metadata) to determine changes to the metadata (block 254 in FIG. 5). The comparison can be used to determine how many and what type of changes have been made to the metadata. In certain instances, the only metadata that is compared is metadata associated with a change to a rhythm. For example, if a change in beat classifications does not change a rhythm classification, the comparison can result in determining that no rhythm changes have been made. However, if a rhythm classification was made by a user, if a beat classification resulted in a rhythm classification changing, if a start or end time of a rhythm was modified, or if a rhythm was deleted or added, the comparison can determine that one or more rhythm changes have been made. In certain instances, the comparison is based on comparing tables of metadata stored to cache memory of an internet browser on a remote computer.

To help visually identify and track the changes, the UI 200 can display various indicators, which are based on changes to metadata (block 256 in FIG. 5).

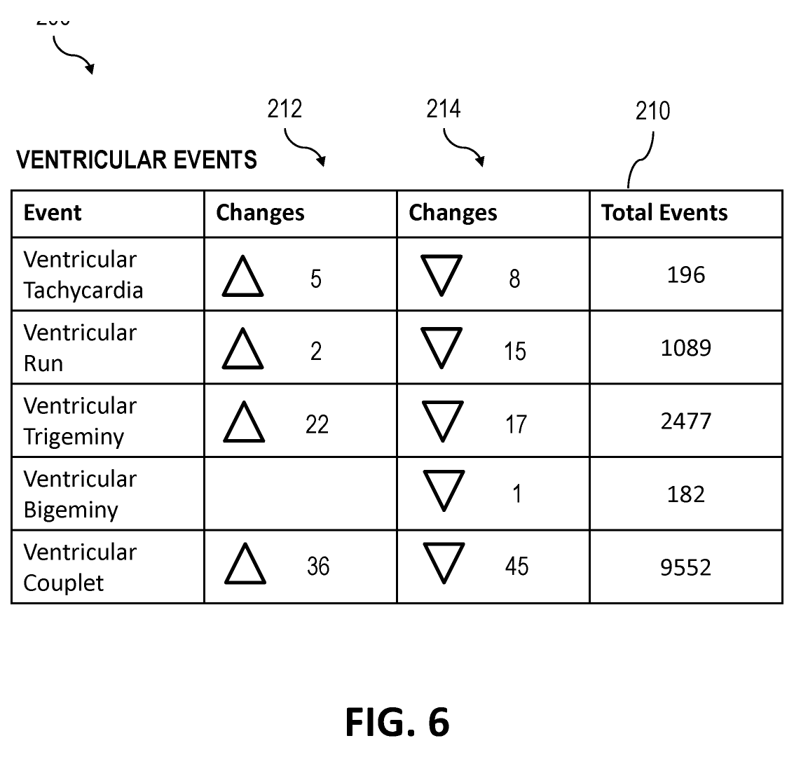

FIG. 6 shows the UI 200 with a window 210 that lists various types of cardiac events. In the example of FIG. 6, the window 210 includes different types of ventricular cardiac events, but it is to be understood that other cardiac events and classes of cardiac events (e.g., atrial fibrillation, pause) can be displayed using the window 210.

Using the top row in the list as an example, the window 210 lists a total number of ventricular tachycardia events within the study being analyzed by a user. Within the same row are two change indicators 212 and 214.

The first change indicator 212 represents the number of regions within the cardiac time series data where rhythms (e.g., ventricular tachycardia rhythms) have been deleted or changed to a different type of rhythms compared to a prior version of the study (e.g., compared to the original metadata generated by the one or more machine learning models). The first change indicator 212 can include a down arrow and number representing the number of modified regions. Further, the first change indicator 212 can be displayed in a certain color (e.g., red) for visual effect.

The second change indicator 214 represents the number of regions within the cardiac time series data where rhythms have been added or changed to that particular type of rhythm compared to a prior version of the study (e.g., compared to the original metadata generated by the one or more machine learning models). For example, if eight NSR rhythms were changed to eight ventricular tachycardia rhythms, the number of the first change indicator would be “8.” The second change indicator 214 can include an up arrow and number representing the number of changes. Further, the second change indicator 214 can be displayed in a certain color (e.g., green) for visual effect.

The change indicators 212 and 214 can also function as icons (with embedded links) that can be selected (via a cursor on the UI 200) to display information about the changed events. For example, one of the change indicators 212, 214 can be selected to display another window in which the user can toggle between changed events. As change indicators 212, 214 are selected or toggled-through, another window or set of windows can display time series cardiac data and metadata such that the changes made can be viewed on the UI 200.

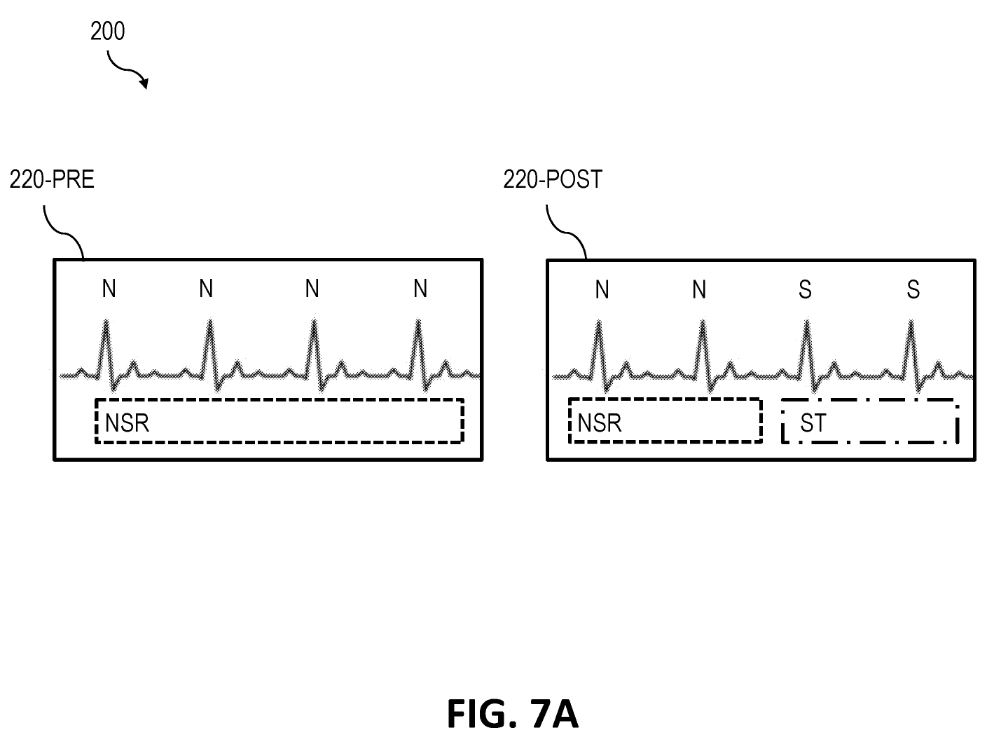

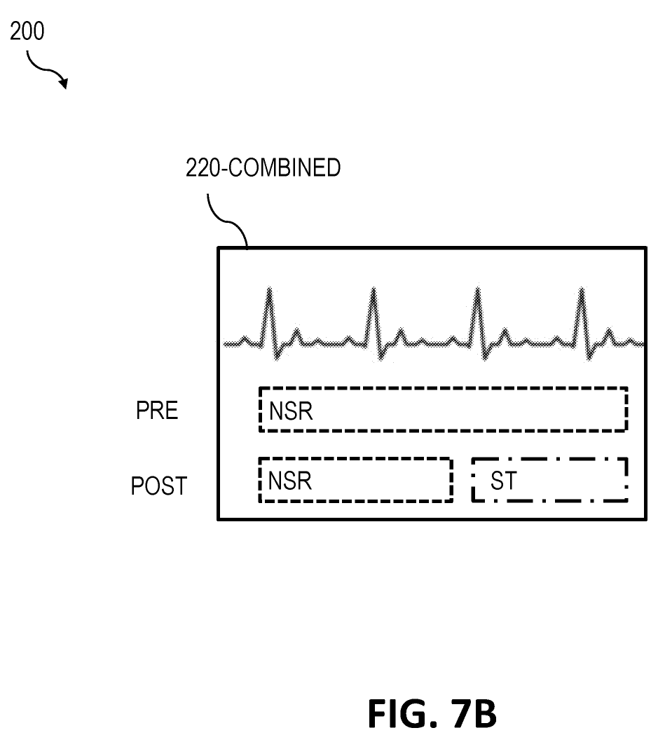

FIGS. 7A and 7B show different examples where a portion of the UI 200 is used to display changes made to the metadata.

In FIG. 7A, the window designated as “220-PRE” displays (i) time series cardiac data, (ii) prior (e.g., original) rhythm classification indicators adjacent to the time series cardiac data, and (iii) prior beat classification indicators adjacent to the time series cardiac data. The window designated as “220-POST” displays the same time series cardiac data as in window 220-PRE. However, the window 220-POST displays updated rhythm classification indicators adjacent to the time series cardiac data and also displays updated (if any) beat classification indicators adjacent to the time series cardiac data.

In the example of FIG. 7A, the last two beats in the displayed time series cardiac data have been changed from normal beats to supraventricular beats. Further, part of the original normal sinus rhythm has been reclassified and changed to a supraventricular tachycardia rhythm classification. As noted herein, the change from a NSR classification to a ST classification could be a result of a user changing certain beats from normal beats to supraventricular beats (which causes the rhythm classification to change) or a user manually changing the classification. Further, as noted herein, the locations of the various indicators can be based, at least in part, on metadata stored to a table.

In FIG. 7B, the window designated as “220-COMBINED” displays (i) time series cardiac data, (ii) prior (e.g., original) rhythm classification indicators adjacent to the time series cardiac data, and (iii) updated rhythm classification indicators adjacent to the time series cardiac data and below the prior rhythm classification indicators such that the changes can be easily viewed using the UI 200.

The examples shown in FIGS. 7A and 7B are just two examples of many other types of rhythm changes that are possible and that can be displayed using the UI 200.

Using the UI 200 described herein, users can select events that have been changed and view the underlying cardiac time series data as well as view side-by-side changes to the classifications of beats and rhythms.

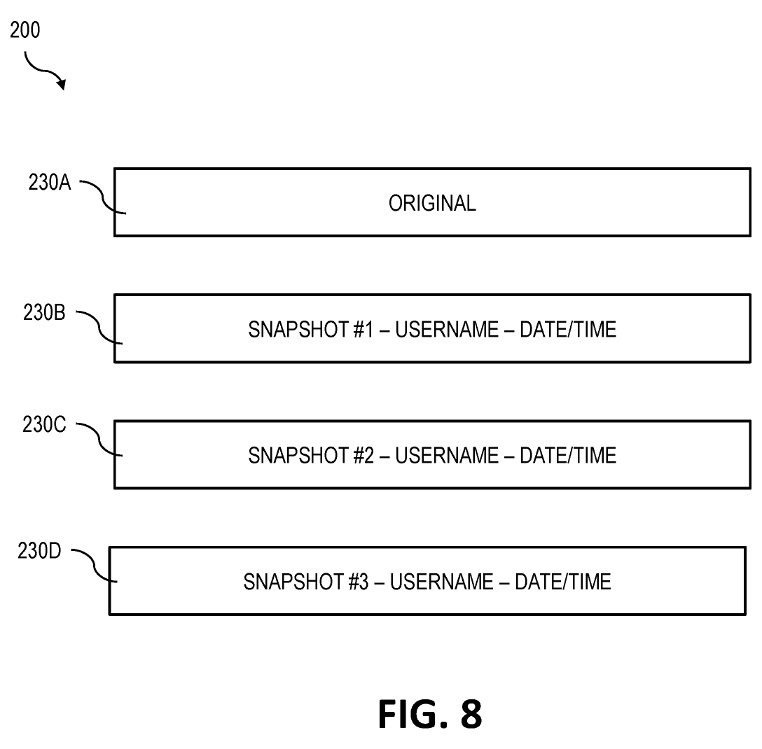

FIG. 8 shows another portion of the UI 200 which helps quickly access changes made to metadata. The UI 200 can display selectable buttons or icons 230A–D that represent different saved versions of the study. Each button 230A–D can be associated with a different table of metadata that was separately saved so that a user can view changes made between different versions of the study. Put another way, each button 230A–D can represent a different snapshot of the study over time. This allows, for example, a more experienced user to audit or review prior changes made by other users at different points in time.

In certain instances, the different versions of the study are saved at the server 106 (FIG. 2) so that users using a different internet browser or using the UI 200 after a prior session has ended can retrieve prior versions of the study. As such, when a user selects one of the buttons 230A–D, the remote computer 116 can send a request to the server 106 to retrieve the table of metadata associated with the selected button. For example, if a user wanted to view the original metadata generated by the one or more machine learning models, the button 230A can be selected to access the time series cardiac data along with the original beat classifications and the original rhythm classifications. If the user wanted to view a later snapshot of the study, the user could select one of the other buttons 230B–D.

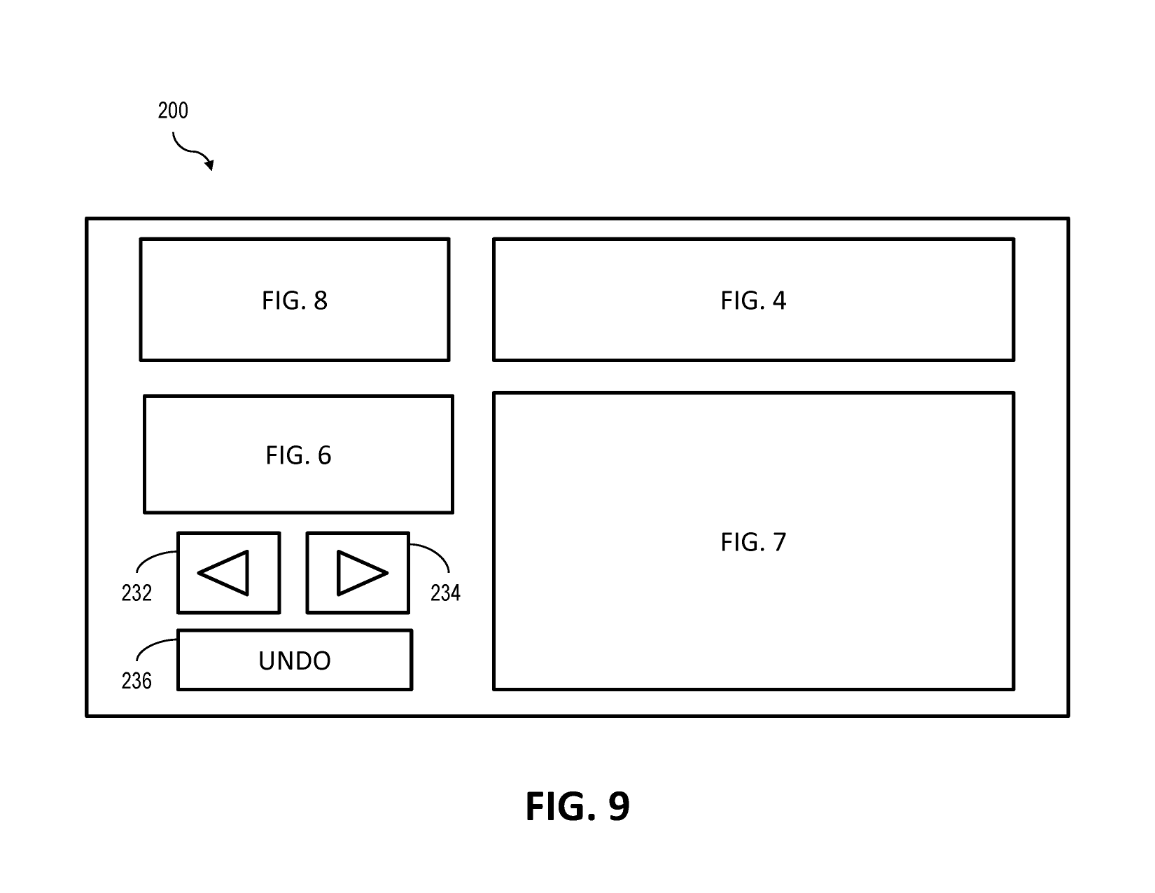

As the user is reviewing prior changes made, the UI 200 can include features for quickly reverting or undoing prior changes to classifications. FIG. 9 shows how the various portions of the UI 200 described herein can be displayed simultaneously in different windows of the UI 200.

As shown in FIG. 9, in addition to the various windows from FIGS. 4 and 6–7, the UI 200 can include buttons 232, 234, and 236 that assist with efficient review of prior changes to metadata. The buttons 232 and 234 can be used to toggle between different changes. For example, if the change indicator 214 displayed in the window 210 of FIG. 6 lists ten changes, the buttons 232 and 234 can be selected to toggle between the ten different changes. As a different change is toggled to, the windows of FIG. 7 can be updated to display the original time series data along with the respective pre-change and post-change beat and/or rhythm classification indicators. This allows a user reviewing prior changes to quickly access and view the changes made.

In the event, a user wants to undo one or more changes, the user can select the button 236 to undo a given change. This allows a user to quickly revert to a prior version of the metadata.

Once the user or users are satisfied with the analysis of the study, a final report can be generated and sent to the patient’s physician. In certain instances, once the report is built and complete, the remote computer 116 can send any changes to the metadata (e.g., the beat classifications, the rhythm classifications, start times, end times) to the server 106 and its database. The server 106 can then replace the metadata initially created by the machine learning model (and saved to the database) with the metadata generated by the remote computer while the user was reviewing and editing the metadata. As such, if the ECG data and metadata need to be accessed again, the server’s database has the most recent version of the metadata. Further, the machine learning model may be further trained on the metadata generated by the user at the remote computer.

### COMPUTING DEVICES AND SYSTEMS

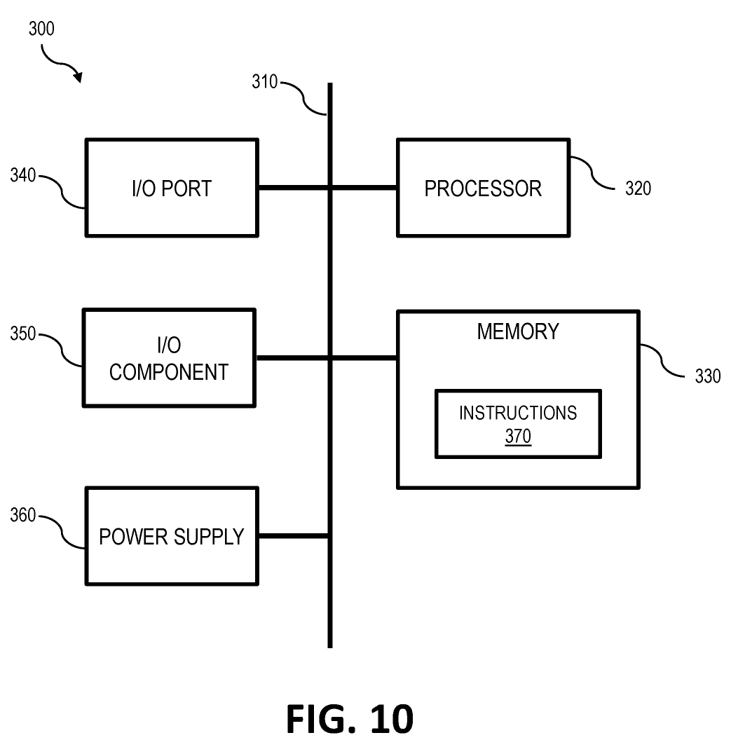

FIG. 10 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 10 and described below. For example, the monitor 102, the mobile device 104, the server 106, and the remote computer 116 can each include their own set of components shown in FIG. 10 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a remote computing system comprising: a user interface, one or more processors, and a non-transitory computer-readable medium having a set of computer-executable instructions configured to be executed by the one or more processors to cause the remote computing system to:
display time series cardiac data on the user interface,
display prior rhythm classification indicators adjacent to the time series cardiac data, wherein respective locations of the prior rhythm classification indicators on the user interface are based, at least in part, on a prior table of metadata,
receive user input, via the user interface, altering the metadata associated with the time series cardiac data,
alter the prior table of metadata to generate an updated table,
compare the updated table to the prior table to determine changes to the metadata,
display a change indicator in a first window on the user interface based on the changes to the metadata, and
display an updated rhythm classification indicator adjacent to the time series cardiac data.

2. The system of claim 1, wherein the remote computing system further includes an internet browser, wherein the prior table and the updated table are stored to cache memory associated with the internet browser.

3. The system of claim 1, wherein the prior table and the updated table comprises a list of cardiac events associated with the time series cardiac data and start and end times for each of the cardiac events.

4. The system of claim 1, wherein the change indicator is a numerical indicator representing a number of changes made to prior rhythm classifications.

5. The system of claim 1, wherein the change indicator is a numerical indicator representing a number of additions or deletions made to prior rhythm classifications.

6. The system of claim 1, wherein the prior rhythm classification indicators are displayed in a second window of the user interface, wherein the updated rhythm classification indicator is displayed in a third window adjacent to the second window.

7. The system of claim 1, wherein the prior rhythm classification indicators and the updated rhythm classification indicator are displayed in a second window of the user interface.

8. The system of claim 1, wherein the updated rhythm classification indicator is a new rhythm classification indicator.

9. The system of claim 1, wherein the updated rhythm classification indicator is longer or shorter than one of the prior rhythm classification indicators.

10. The system of claim 1, wherein the updated rhythm classification indicator is a different type of cardiac event from one of the prior rhythm classifications.

11. The system of claim 1, wherein the updated table comprises the updated rhythm classification in place of at least one of the prior rhythm classifications.

12. A method comprising:
displaying time series cardiac data on a user interface;
displaying prior rhythm classification indicators adjacent to the time series cardiac data, wherein respective locations of the prior rhythm classification indicators on the user interface are based, at least in part, on a prior table of metadata;
receiving user input, via the user interface, altering the metadata associated with the time series cardiac data;
altering the prior table of metadata to generate an updated table;
comparing the updated table to the prior table to determine changes to the metadata;
displaying a change indicator in a first window on the user interface based on the changes to the metadata; and
displaying an updated rhythm classification indicator adjacent to the time series cardiac data.

13. The method of claim 12, wherein the prior table and the updated table are stored to cache memory of a browser.

14. The method of claim 12, wherein the prior table and the updated table comprises a list of cardiac events associated with the time series cardiac data and start and end times for each of the cardiac events.

15. The method of claim 12, wherein the change indicator is a numerical indicator representing a number of changes made to prior rhythm classifications.

16. The method of claim 12, wherein the change indicator is a numerical indicator representing a number of additions or deletions made to prior rhythm classifications.

17. The method of claim 12, wherein the altering comprises adding a new rhythm event, wherein the updated rhythm classification indicator is a new rhythm classification indicator.

18. The method of claim 12, wherein the altering comprises changing a rhythm start time or a rhythm end time, wherein the updated rhythm classification indicator is longer or shorter than one of the prior rhythm classification indicators.

19. The method of claim 12, wherein the altering comprises changing a beat classification, wherein the updated rhythm classification indicator is a different type of cardiac event from one of the prior rhythm classifications.

20. The method of claim 12, wherein the altering comprises changing one of the prior rhythm classifications to the updated rhythm classification.
